(19) United States
(12) Patent Application Publication
(10) Pub. No.: US 2013/0331445 A1
(43) Pub. Date: Dec. 12, 2013

(54) Picropodophyllin Monohydrate or Polymorph A in Cancer Therapy

(71) Applicants: Mikael Bisrat, Strangnas (SE); Magnus Brisander, Ekero (SE)

(72) Inventors: Mikael Bisrat, Strangnas (SE); Magnus Brisander, Ekero (SE)

(73) Assignee: Axelar AB, Soina (SE)

(21) Appl. No.: 13/877,990

(22) PCT Filed: Oct. 7, 2011
(86) PCT No.: PCT/SE11/51208

§ 371 Date: Aug. 13, 2013

Related U.S. Application Data
(60) Provisional application No. 61/391,108, filed on Oct. 8, 2010.
(60) Provisional application No. 61/410,014, filed on Nov. 4, 2010.

(51) Int. Cl. A61K 31/365

(52) U.S. Cl. CPC A61K 31/365
USPC 514/463

(57) ABSTRACT

The invention related to picropodophyllin monohydrate as well as to picropodophyllin polymorph A for use in therapy, such as their use in cancer therapy.

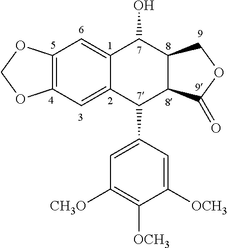

### FIELD OF THE INVENTION

The present invention relates to picropodophyllin monohydrate as well as to picropodophyllin polymorph A, for use in therapy.

### BACKGROUND OF THE INVENTION

Pharmaceutical solids can exist in different forms, such as crystalline, amorphous, or glass and also in solvated or hydrated forms. A polymorph is a solid crystalline phase of a compound resulting from the possibility of at least two crystalline arrangements of the molecules of that compound in the solid state.

It is a well known fact that different forms of the same drug may provide differences in certain pharmaceutically important physicochemical properties, such as stability, solubility, dissolution rate, crystal habit and tableting behavior. Changes in certain of these physiochemical properties may ultimately affect the bioavailability of the drug.

Picropodophyllin is a compound belonging to the class of compounds denominated cyclolignans, having the chemical structure:

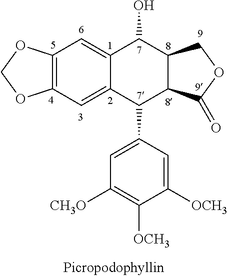

For a long time, picropodophyllin attracted little interest, since it was believed to possess no or low biological activity. In contrast, its stereoisomer podophyllotoxin, which has a trans configuration in the lactone ring, has been studied for decades due to its cytotoxic properties.

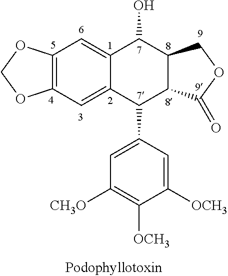

However, research has proven that picropodophyllin exhibits interesting biological properties and hence potential as a medicament.

WO 02/102804 discloses that picropodophyllin is a specific and potent inhibitor of insulin-like growth factor-1 receptor (IGF-1R) and may be useful in the treatment of IGF-1R dependent diseases such as various types of cancer, artheriosclerosis, psoriasis, and restenosis following coronary angioplasty.

WO 2007/097707 discloses the use of picropodophyllin in the prophylaxis or treatment of diabetes mellitus type 2, nephropathy, retinopathy, macular degeneration, retinopathy of prematurity, central retinal vein occlusion, branch retinal vein occlusion, rubeotic glaucoma, thyroid eye disease, corneal graft rejection and corneal chemical burns; and for contraception.

WO 2009/157858 discloses the use of picropodophyllin for the prophylaxis or treatment of diseases or conditions characterized by a hyperactive immune system such as rheumatoid arthritis, Crohn's disease, ulcerative colitis, multiple sclerosis, Alzheimer's disease, asthma, eczematous dermatitis, and graft rejection following transplantation.

Z. Kristallogr. 215 (2000), pp. 45-47, discloses a crystalline structure of picropodophyllin for which crystal data are reported.

Picropodophyllin monohydrate and picropodophyllin polymorph A are disclosed by Schrecker et al in Helvetica Chimica Acta (1954); 37; pp. 1541-1543.

### DESCRIPTION OF THE INVENTION

### Brief Description of the Accompanying Drawings

FIG. 1 is an X-ray powder diffractogram (XRPD) of picropodophyllin monohydrate, measured on a zero background quarts single crystal specimen support.

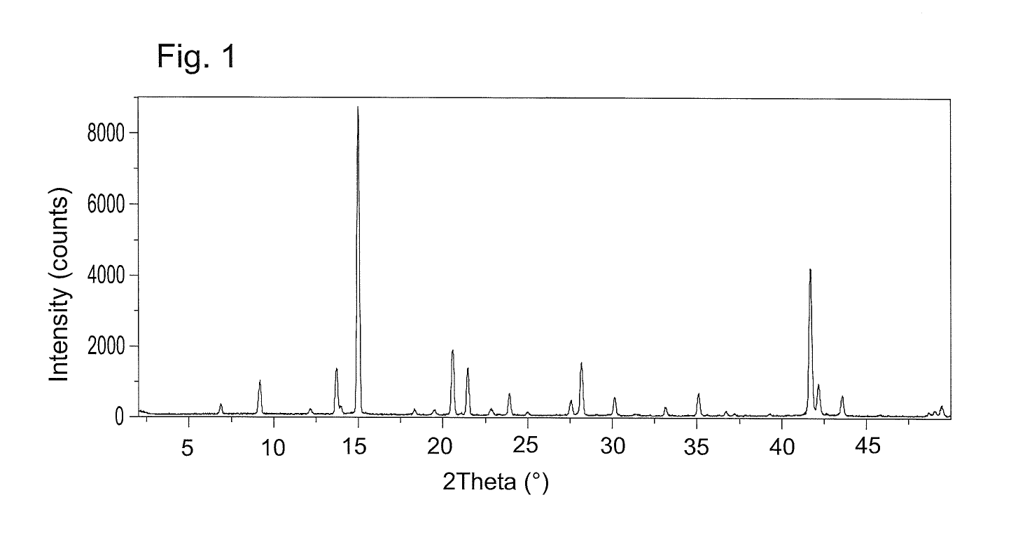

FIG. 2 is an X-ray powder diffractogram (XRPD) of picropodophyllin polymorph A, measured on a zero background quarts single crystal specimen support.

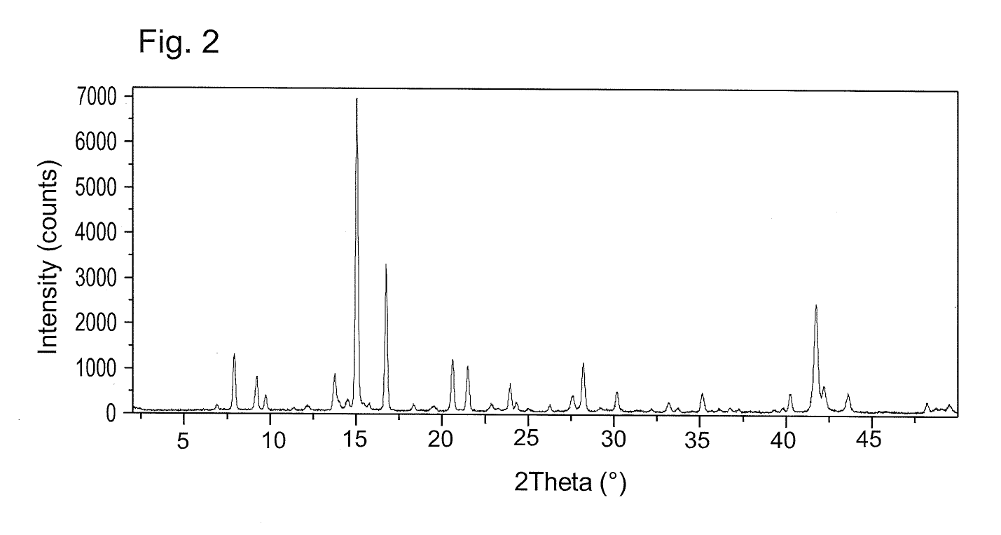

An aspect of the present invention is picropodophyllin monohydrate for use in therapy.

An aspect of the present invention is picropodophyllin polymorph A for use in therapy.

Picropodophyllin monohydrate as herein described, has good physiochemical and solid state properties for pharmaceutical product development.

Still an aspect of the invention is picropodophyllin monohydrate for use in therapy, said picropodophyllin monohydrate having physiochemical and solid state properties making it suitable in the preparation of suspensions for medical use.

Still an aspect of the invention is picropodophyllin monohydrate for use in therapy, said picropodophyllin monohydrate having good shelf life stability.

One aspect of the present invention, is picropodophyllin monohydrate characterized by having an X-ray powder diffraction pattern exhibiting a peak at 6.9±0.2°2θ, for use in therapy.

One aspect of the invention, is picropodophyllin monohydrate characterized by having an X-ray powder diffraction pattern exhibiting peaks at 6.9 and 9.2±0.2°2θ, for use in therapy.

Yet an aspect of the invention is picropodophyllin monohydrate characterized by having an X-ray powder diffraction pattern exhibiting peaks at 6.9, 9.2, 13.7 and 15.0±0.2°2θ, for use in therapy.

One aspect of the invention, is picropodophyllin monohydrate characterized by having an X-ray powder diffraction pattern exhibiting peaks at 6.9, 9.2, 13.7, 15.0, 20.6 and 21.5±0.2°2θ, for use in therapy.

A further aspect of the invention, is picropodophyllin monohydrate characterized by having an X-ray powder diffraction pattern exhibiting a peak at 9.2±0.2°2θ, for use in therapy.

One aspect of the invention, is picropodophyllin monohydrate characterized by having an X-ray powder diffraction pattern exhibiting peaks at 9.2 and 13.7±0.2°2θ, for use in therapy.

One aspect of the invention, is picropodophyllin monohydrate characterized by having an X-ray powder diffraction pattern exhibiting peaks at 9.2, 13.7, 15.0, 20.6 and 21.5±0.2°2θ, for use in therapy.

Still an aspect of the invention is picropodophyllin monohydrate as herein defined, substantially free from polymorphs and/or other crystal and non-crystal forms of picropodophyllin, for use in therapy.

The wording “substantially free from polymorphs and/or other crystal and non-crystal forms of picropodophyllin” shall be understood to mean that picropodophyllin monohydrate contains less than 10%, such as less than 5%, or less than 1% of any polymorph and/or other crystal and non-crystal forms of picropodophyllin.

An aspect of the present invention is picropodophyllin polymorph A, for use in therapy.

An aspect of the invention, is picropodophyllin polymorph A characterized by having an X-ray powder diffraction pattern exhibiting a peak at 6.9±0.2°2θ, for use in therapy.

An aspect of the invention, is picropodophyllin polymorph A characterized by having an X-ray powder diffraction pattern exhibiting peaks at 6.9 and 7.9±0.2°2θ, for use in therapy.

Yet an aspect of the invention, is picropodophyllin polymorph A characterized by having an X-ray powder diffraction pattern exhibiting peaks at 6.9, 7.9, 9.2 and 9.7±0.2°2θ, for use in therapy.

Still an aspect of the invention, is picropodophyllin polymorph A characterized by having an X-ray powder diffraction pattern exhibiting peaks at 6.9, 7.9, 9.2, 9.7, 15.0 and 16.7±0.2°2θ, for use in therapy.

One aspect of the invention is picropodophyllin polymorph A substantially free from other polymorphs and/or other crystal and non-crystal forms of picropodophyllin, for use in therapy.

The wording “substantially free from other polymorphs and/or other crystal and non-crystal forms of picropodophyllin” shall be understood to mean that picropodophyllin polymorph A contains less than 10%, such as less than 5%, or less than 1% of any polymorphs and/or other crystal and non-crystal forms of picropodophyllin.

Still an aspect of the invention, is the use of picropodophyllin monohydrate as herein defined, for the manufacture of a medicament for the treatment of IGF-1R dependent diseases such as cancer.

Yet an aspect of the invention is the use of picropodophyllin monohydrate as herein defined, for the manufacture of a medicament for the treatment of lung cancer such as non-small cell lung cancer (NSCLC) or small cell lung cancer; breast cancer; head and neck cancer such as oral, sinusoidal or pharyngeal cancer; gastrointestinal cancer such as oesophageal cancer, stomach cancer, colon cancer, rectal cancer, gastrointestinal stromal tumor, liver cancer or pancreatic cancer; genitourinary cancer such as prostate cancer, bladder cancer or kidney cancer; gynecologic cancer such as ovarian cancer, cervical cancer, endometric cancer or uterine sarcoma; hematologic cancer such as myeloid leukemia, lymphocytic leukemia, lymphomas or multiple myeloma; musculoskeletal cancer such as Ewings sarcoma, osteosarcoma or soft tissue sarcoma; skin cancer such as malignant melanoma, basal cell cancer, squamous cell cancer or Kaposi's sarcoma; brain and neurologic cancer such as gliomas, glioblastoma, astrocytoma, medulloblastoma, craniopharyngeoma or neuroblastoma; endocrine cancer such as adrenocortical cancer, paraganglioma, pheochromocytoma or thyroid cancer; or eye cancer such as retinoblastoma or uveal melanoma.

Examples of non-small cell lung cancer (NSCLC) where picropodophyllin monohydrate as herein defined may be useful, are adenocarcinoma, squameous, or large-cell carcinoma.

Yet an aspect of the present invention is the use of picropodophyllin monohydrate as herein defined, for the manufacture of a medicament for the treatment of psoriasis; restenosis after coronary angioplasty; diabetes mellitus type 2; nephropathy; eye diseases such as retinopathy or macular degeneration; rheumatoid arthritis; inflammatory bowel disease such as Crohns disease or ulcerative colitis; multiple sclerosis; Alzheimers disease; or graft rejection.

Still an aspect of the invention, is picropodophyllin monohydrate as herein defined, for use in the treatment of IGF-1R dependent diseases such as cancer.

Yet an aspect of the invention, is picropodophyllin monohydrate as herein defined, for use in the treatment of lung cancer such as non-small cell lung cancer (NSCLC) or small cell lung cancer; breast cancer; head and neck cancer such as oral, sinusoidal or pharyngeal cancer; gastrointestinal cancer such as oesophageal cancer, stomach cancer, colon cancer, rectal cancer, gastrointestinal stromal tumor, liver cancer or pancreatic cancer; genitourinary cancer such as prostate cancer, bladder cancer or kidney cancer; gynecologic cancer such as ovarian cancer, cervical cancer, endometric cancer or uterine sarcoma; hematologic cancer such as myeloid leukemia, lymphocytic leukemia, lymphomas or multiple myeloma; musculoskeletal cancer such as Ewings sarcoma, osteosarcoma or soft tissue sarcoma; skin cancer such as malignant melanoma, basal cell cancer, squamous cell cancer or Kaposi's sarcoma; brain and neurologic cancer such as gliomas, glioblastoma, astrocytoma, medulloblastoma, craniopharyngeoma or neuroblastoma; endocrine cancer such as adrenocortical cancer, paraganglioma, pheochromocytoma or thyroid cancer; or eye cancer such as retinoblastoma or uveal melanoma.

Yet an aspect of the invention, is picropodophyllin monohydrate as herein defined, for use in the treatment of psoriasis; restenosis after coronary angioplasty; diabetes mellitus type 2; nephropathy; eye diseases such as retinopathy or macular degeneration; rheumatoid arthritis; inflammatory bowel disease such as Crohns disease or ulcerative colitis; multiple sclerosis; Alzheimers disease; or graft rejection.

One aspect of the invention is a method for the treatment of IGF-1R dependent diseases such as cancer, comprising the administration of a therapeutically effective amount of picropodophyllin monohydrate as herein defined, to a patient in need of such treatment. Still an aspect of the invention is a method for the treatment of lung cancer such as non-small cell lung cancer (NSCLC) or small cell lung cancer; breast cancer; head and neck cancer such as oral, sinusoidal or pharyngeal cancer; gastrointestinal cancer such as oesophageal cancer, stomach cancer, colon cancer, rectal cancer, gastrointestinal stromal tumor, liver cancer or pancreatic cancer; genitourinary cancer such as prostate cancer, bladder cancer or kidney cancer; gynecologic cancer such as ovarian cancer, cervical cancer, endometric cancer or uterine sarcoma; hematologic cancer such as myeloid leukemia, lymphocytic leukemia, lymphomas or multiple myeloma; musculoskeletal cancer such as Ewings sarcoma, osteosarcoma or soft tissue sarcoma; skin cancer such as malignant melanoma, basal cell cancer, squamous cell cancer or Kaposi's sarcoma; brain and neurologic cancer such as gliomas, glioblastoma, astrocytoma, medulloblastoma, craniopharyngeoma or neuroblastoma; endocrine cancer such as adrenocortical cancer, paraganglioma, pheochromocytoma or thyroid cancer; or eye cancer such as retinoblastoma or uveal melanoma; comprising the administration of a therapeutically effective amount of picropodophyllin monohydrate as herein defined, to a patient in need of such treatment.

One aspect of the invention is a method for the treatment of psoriasis; restenosis after coronary angioplasty; diabetes mellitus type 2; nephropathy; eye diseases such as retinopathy or macular degeneration; rheumatoid arthritis; inflammatory bowel disease such as Crohns disease or ulcerative colitis; multiple sclerosis; Alzheimers disease; or graft rejection; comprising the administration of a therapeutically effective amount of picropodophyllin monohydrate as herein defined, to a patient in need of such treatment.

Still an aspect of the invention, is the use of picropodophyllin polymorph A as herein defined, for the manufacture of a medicament for the treatment of IGF-1R dependent diseases such as cancer.

Yet an aspect of the invention is the use of picropodophyllin polymorph A as herein defined, for the manufacture of a medicament for the treatment of lung cancer such as non-small cell lung cancer (NSCLC) or small cell lung cancer; breast cancer; head and neck cancer such as oral, sinusoidal or pharyngeal cancer; gastrointestinal cancer such as oesophageal cancer, stomach cancer, colon cancer, rectal cancer, gastrointestinal stromal tumor, liver cancer or pancreatic cancer; genitourinary cancer such as prostate cancer, bladder cancer or kidney cancer; gynecologic cancer such as ovarian cancer, cervical cancer, endometric cancer or uterine sarcoma; hematologic cancer such as myeloid leukemia, lymphocytic leukemia, lymphomas or multiple myeloma; musculoskeletal cancer such as Ewings sarcoma, osteosarcoma or soft tissue sarcoma; skin cancer such as malignant melanoma, basal cell cancer, squamous cell cancer or Kaposi's sarcoma; brain and neurologic cancer such as gliomas, glioblastoma, astrocytoma, medulloblastoma, craniopharyngeoma or neuroblastoma; endocrine cancer such as adrenocortical cancer, paraganglioma, pheochromocytoma or thyroid cancer; or eye cancer such as retinoblastoma or uveal melanoma.

Examples of non-small cell lung cancer (NSCLC) where picropodophyllin polymorph A as herein defined may be useful, are adenocarcinoma, squameous, or large-cell carcinoma.

Yet an aspect of the present invention is the use of picropodophyllin polymorph A as herein defined, for the manufacture of a medicament for the treatment of psoriasis; restenosis after coronary angioplasty; diabetes mellitus type 2; nephropathy; eye diseases such as retinopathy or macular degeneration; rheumatoid arthritis; inflammatory bowel disease such as Crohns disease or ulcerative colitis; multiple sclerosis; Alzheimers disease; or graft rejection.

Still an aspect of the invention, is picropodophyllin polymorph A as herein defined, for use in the treatment of IGF-1R dependent diseases such as cancer.

Yet an aspect of the invention, is picropodophyllin polymorph A as herein defined, for use in the treatment of lung cancer such as non-small cell lung cancer (NSCLC) or small cell lung cancer; breast cancer; head and neck cancer such as oral, sinusoidal or pharyngeal cancer; gastrointestinal cancer such as oesophageal cancer, stomach cancer, colon cancer, rectal cancer, gastrointestinal stromal tumor, liver cancer or pancreatic cancer; genitourinary cancer such as prostate cancer, bladder cancer or kidney cancer; gynecologic cancer such as ovarian cancer, cervical cancer, endometric cancer or uterine sarcoma; hematologic cancer such as myeloid leukemia, lymphocytic leukemia, lymphomas or multiple myeloma; musculoskeletal cancer such as Ewings sarcoma, osteosarcoma or soft tissue sarcoma; skin cancer such as malignant melanoma, basal cell cancer, squamous cell cancer or Kaposi's sarcoma; brain and neurologic cancer such as gliomas, glioblastoma, astrocytoma, medulloblastoma, craniopharyngeoma or neuroblastoma; endocrine cancer such as adrenocortical cancer, paraganglioma, pheochromocytoma or thyroid cancer; or eye cancer such as retinoblastoma or uveal melanoma.

Yet an aspect of the invention, is picropodophyllin polymorph A as herein defined, for use in the treatment of psoriasis; restenosis after coronary angioplasty; diabetes mellitus type 2; nephropathy; eye diseases such as retinopathy or macular degeneration; rheumatoid arthritis; inflammatory bowel disease such as Crohns disease or ulcerative colitis; multiple sclerosis; Alzheimers disease; or graft rejection.

One aspect of the invention is a method for the treatment of IGF-1R dependent diseases such as cancer, comprising the administration of a therapeutically effective amount of picropodophyllin polymorph A as herein defined, to a patient in need of such treatment.

Still an aspect of the invention is a method for the treatment of lung cancer such as non-small cell lung cancer (NSCLC) or small cell lung cancer; breast cancer; head and neck cancer such as oral, sinusoidal or pharyngeal cancer; gastrointestinal cancer such as oesophageal cancer, stomach cancer, colon cancer, rectal cancer, gastrointestinal stromal tumor, liver cancer or pancreatic cancer; genitourinary cancer such as prostate cancer, bladder cancer or kidney cancer; gynecologic cancer such as ovarian cancer, cervical cancer, endometric cancer or uterine sarcoma; hematologic cancer such as myeloid leukemia, lymphocytic leukemia, lymphomas or multiple myeloma; musculoskeletal cancer such as Ewings sarcoma, osteosarcoma or soft tissue sarcoma; skin cancer such as malignant melanoma, basal cell cancer, squamous cell cancer or Kaposi's sarcoma; brain and neurologic cancer such as gliomas, glioblastoma, astrocytoma, medulloblastoma, craniopharyngeoma or neuroblastoma; endocrine cancer such as adrenocortical cancer, paraganglioma, pheochromocytoma or thyroid cancer; or eye cancer such as retinoblastoma or uveal melanoma; comprising the administration of a therapeutically effective amount of picropodophyllin polymorph A as herein defined, to a patient in need of such treatment.

One aspect of the invention is a method for the treatment of psoriasis; restenosis after coronary angioplasty; diabetes mellitus type 2; nephropathy; eye diseases such as retinopathy or macular degeneration; rheumatoid arthritis; inflammatory bowel disease such as Crohns disease or ulcerative colitis; multiple sclerosis; Alzheimers disease; or graft rejection; comprising the administration of a therapeutically effective amount of picropodophyllin polymorph A as herein defined, to a patient in need of such treatment.

One aspect of the invention, is the use of a pharmaceutical composition comprising picropodophyllin monohydrate or picropodophyllin polymorph A as herein described, in admixture with a pharmaceutically acceptable adjuvant, diluent and/or carrier.

Still an aspect of the invention is the use of at least one anti-cancer drug, in combination with picropodophyllin monohydrate as herein described, or in combination with picropodophyllin polymorph A as herein described.

Still an aspect of the invention is the use of at least one anti-cancer drug in combination with picropodophyllin monohydrate as herein described, or in combination with picropodophyllin polymorph A as herein described, wherein the at least one anti-cancer drug and picropodophyllin monohydrate or picropodophyllin polymorph A, are administered sequentially, separately or simultaneously to a patient in need thereof.

In one aspect of the invention, there is provided a kit of parts comprising:

(i) picropodophyllin monohydrate, or picropodophyllin polymorph A; and
(ii) an anti-cancer drug;
for sequential, separate or simultaneous administration.

In one aspect of the invention, there is provided a kit of parts as herein described, for use in therapy.

Yet an aspect of the invention is a kit of parts as herein described, for the treatment of cancer such as lung non-small cell lung cancer (NSCLC) or small cell lung cancer; breast cancer; head and neck cancer such as oral, sinusoidal or pharyngeal cancer; gastrointestinal cancer such as oesophageal cancer, stomach cancer, colon cancer, rectal cancer, gastrointestinal stromal tumor, liver cancer or pancreatic cancer; genitourinary cancer such as prostate cancer, bladder cancer or kidney cancer; gynecologic cancer such as ovarian cancer, cervical cancer, endometric cancer or uterine sarcoma; hematologic cancer such as myeloid leukemia, lymphocytic leukemia, lymphomas or multiple myeloma; musculoskeletal cancer such as Ewings sarcoma, osteosarcoma or soft tissue sarcoma; skin cancer such as malignant melanoma, basal cell cancer, squamous cell cancer or Kaposi's sarcoma; brain and neurologic cancer such as gliomas, glioblastoma, astrocytoma, medulloblastoma, craniopharyngeoma or neuroblastoma; endocrine cancer such as adrenocortical cancer, paraganglioma, pheochromocytoma or thyroid cancer; or eye cancer such as retinoblastoma or uveal melanoma.

Picropodophyllin monohydrate as herein described, may be administered via the oral, parenteral, intravenous, intramuscular, subcutaneous or by injectable administration routes, buccal, rectal, vaginal, transdermal, nasal or ophtalmic route, or via inhalation in the form of pharmaceutical compositions comprising a pharmaceutically acceptable dosage form. Depending upon the disorder and patient to be treated and the route of administration, the compositions may be administered at varying doses. In one aspect of the invention, picropodophyllin monohydrate as herein described, is present in an amount of 1-95% by weight of the total weight of the pharmaceutical composition.

Picropodophyllin polymorph A as herein described, may be administered via the oral, parenteral, intravenous, intramuscular, subcutaneous or by injectable administration routes, buccal, rectal, vaginal, transdermal, nasal or ophtalmic route, or via inhalation in the form of pharmaceutical compositions comprising a pharmaceutically acceptable dosage form. Depending upon the disorder and patient to be treated and the route of administration, the compositions may be administered at varying doses. In one aspect of the invention, picropodophyllin monohydrate as herein described, is present in an amount of 1-95% by weight of the total weight of the pharmaceutical composition.

An aspect of the present invention is the use of a pharmaceutical composition comprising picropodophyllin monohydrate as herein described, in admixture with a pharmaceutically and pharmacologically acceptable adjuvant and/or carrier. The pharmaceutically and pharmacologically acceptable carrier suitable for a particular pharmaceutical composition will be apparent to a person skilled in the art of pharmaceutical compositions. The pharmaceutical composition may be administered to a subject or patient by an administration route suitable for the type of cancer or medical indication to be treated. For parenteral administration, picropodophyllin monohydrate as herein described, may be administered as an injectable dosage form, by continuous infusion which may be intravenous, as a solution or as a suspension.

For oral administration, picropodophyllin monohydrate as herein described, may be administered as a capsule comprising said picropodophyllin monohydrate as herein described, in form of a suspension, or as a solution.

In one aspect of the present invention, the dosage of picropodophyllin monohydrate or picropodophyllin polymorph A as herein described, may range from 1-40 mg/kg body weight per day.

In one aspect of the present invention, picropodophyllin monohydrate or picropodophyllin polymorph A as herein described, is administered in a dosage of 400 mg twice daily.

In yet an aspect of the present invention, picropodophyllin monohydrate or picropodophyllin polymorph A as herein described, is administered in a dosage of 390 mg twice daily.

In one aspect of the invention, picropodophyllin monohydrate as herein described, is administered as an oral suspension.

In yet an aspect of the invention, picropodophyllin monohydrate as herein described, is administered as an oral suspension comprising 25 mg/ml of picropodophyllin monohydrate as herein described.

Still an aspect of the invention is the use of a combination of at least one anti-cancer drug and picropodophyllin monohydrate, or picropodophyllin polymorph A, as herein described.

Examples of anti-cancer drugs useful in combination with picropodophyllin monohydrate or picropodophyllin polymorph A as herein described are cytostatics; targeted anticancer agents being monoclonal antibodies or selective small-molecule inhibitors; hormones; antihormones; or immunostimulating agents.

Examples of cytostatics useful in combination therapy with picropodophyllin monohydrate or picropodophyllin polymorph A, as herein described, are alkylating agents such as melphalan; antimetabolites such as methotrexate or gemcitabine; mitotic inhibitors such as taxanes or vinca alkaloids; cytotoxic antibiotics such as doxorubicin; topoisomerase II inhibitors such as etoposide; or other cytostatics such as cisplatin or carboplatin.

Examples of monoclonal antibodies useful in combination therapy with picropodophyllin monohydrate or picropodophyllin polymorph A, as herein described, are those targeting the epidermal growth factor receptor (EGFR), HER2, or vascular endothelial growth factor such as trastozumab or bevacizumab.

Examples of selective small-molecule inhibitors useful in combination therapy with picropodophyllin monohydrate or picropodophyllin polymorph A, as herein described, are those targeting epidermal growth factor receptor, histone deacetylase (HDAC), Raf, platelet-derived growth factor receptors, vascular endothelial growth factor receptor, or c-Kit, such as gefitinib or imatinib.

Examples of hormones useful in combination therapy with picropodophyllin monohydrate or picropodophyllin polymorph A, as herein described, are estrogens or gestagens.

Examples of antihormones useful in combination therapy with picropodophyllin monohydrate or picropodophyllin polymorph A, as herein described, are antiestrogens, antiandrogens or enzyme inhibitors.

Examples of immunostimulating agents useful in combination therapy with picropodophyllin monohydrate or picropodophyllin polymorph A, as herein described, are interferons.

All of the preceding aspects may also be used with any claims, aspects or embodiments of the invention hereinbefore or hereinafter.

In one aspect of the invention, there is provided a kit of parts comprising:
(i) picropodophyllin monohydrate as herein described, or picropodophyllin polymorph A as herein described, and
(ii) (ii) an anti-cancer drug; for the sequential, separate or simultaneous administration of picropodophyllin monohydrate or picropodophyllin polymorph A as herein described.

In one aspect of the invention, there is provided the use of a kit of parts as herein defined for the manufacture of a medicament for the treatment of IGF-1R dependent diseases such as cancer.

Yet an aspect of the invention is the use of a kit of parts as herein defined for the manufacture of a medicament for the treatment of lung cancer such as non-small cell lung cancer (NSCLC) or small cell lung cancer; breast cancer; head and neck cancer such as oral, sinusoidal or pharyngeal cancer; gastrointestinal cancer such as oesophageal cancer, stomach cancer, colon cancer, rectal cancer, gastrointestinal stromal tumor, liver cancer or pancreatic cancer; genitourinary cancer such as prostate cancer, bladder cancer or kidney cancer; gynecologic cancer such as ovarian cancer, cervical cancer, endometric cancer or uterine sarcoma; hematologic cancer such as myeloid leukemia, lymphocytic leukemia, lymphomas or multiple myeloma; musculoskeletal cancer such as Ewings sarcoma, osteosarcoma or soft tissue sarcoma; skin cancer such as malignant melanoma, basal cell cancer, squamous cell cancer or Kaposi's sarcoma; brain and neurologic cancer such as gliomas, glioblastoma, astrocytoma, medulloblastoma, craniopharyngeoma or neuroblastoma; endocrine cancer such as adrenocortical cancer, paraganglioma, pheochromocytoma or thyroid cancer; or eye cancer such as retinoblastoma or uveal melanoma.

All of the preceding aspects may also be used with any claims, aspects or embodiments of the invention hereinbefore or hereinafter.

### Methods of Preparation

Picropodophyllin monohydrate as herein defined, is prepared by:
a) adding an aqueous solution of a base to a solution of podophyllotoxin in a protic solvent,
b) heating the reaction mixture from step a) to a temperature of between 70 and 75° C. for at least 2 hours,
c) cooling the reaction mixture from step b),
d) isolating the product.
e) washing the product with a solvent,
f) drying the product, and
g) conditioning the product with water.

In a further aspect, the base in step a) may be NaOAc.

In a further aspect, the protic solvent in step a) may be ethanol.

In a further aspect, step d) may be performed using a filter.

In a further aspect, step e) may be performed with ethanol.

In a further aspect, step f) may be performed under vacuum.

Picropodophyllin monohydrate was obtained as described in Example 1.

### ABBREVIATIONS

DVS Dynamic Vapor Sorption
LC Liquid chromatography
LC UV Liquid chromatography Ultraviolet Spectroscopy
ml milliliter
L Liter
PVDF Polyvinylidene fluoride
RH Relative Humidity
SDS sodium dodecyl sulfate
XRPD X-ray powder diffraction

### EXAMPLES

### X-Ray Powder Diffraction (XRPD)

X-Ray Powder Diffraction (XRPD) experiments were run on an X'Pert Pro diffractometer (PANanalytical B.V., Netherlands) set in Bragg-Brentano geometry. The diffractometer was equipped with a Ge(111) primary monochromator and PIXcell detector. A representative sample was placed on a zero background quarts single crystal specimen support (Siltronix, France).

Experiments were run using Cu K_{α1} radiation (45 kV and 40 mA) at ambient temperature and humidity. Scans were run in continuos scan mode in the range 2-50° 2θ using automatic divergence and antiscatter slits with observed length of 10 mm, a step size of 0.0131° 2θ and a common counting time of 217.770 seconds.

It will be understood by a person skilled in the art, that the 2-theta values of the X-ray powder diffraction pattern may vary slightly from one machine to another. Some variation may also exist due to sample preparation and variations between batches.

Data collections were done with the application software X'Pert Data Collector version 2.2d and instrument control software version 1.9D, and pattern analysis and profile refinement was done with X'Pert HighScore Plus version 2.2.3. All software's comes from PANanalytical B.V., Netherlands.

### Example 1

### Preparation of Picropodophyllin Monohydrate

17.3 kg (127 moles) of NaOAc×3H₂O was dissolved in water, filtered and added to a filtered solution of 10.5 kg (25 moles) of picropodophyllin in ethanol (198 L). The reaction mixture was kept at 70-75° C. during at least 2 hours, whereafter it was cooled. The product picropodophyllin was isolated through a Nutch filter, washed with ethanol (at least 50%) and dried under vacuum. The thus obtained product was subjected to conditioning with water during at least 96 hours to yield picropodophyllin monohydrate (8 kg).

### XRPD Peak Positions

### Picropodophyllin Monohydrate

### Refined 2θ Values:

6.9±0.2°2θ
9.2±0.2°2θ
13.7±0.2°2θ
15.0±0.2°2θ
20.6±0.2°2θ
21.5±0.2°2θ

### Example 2

### Solubility Studies for Picropodophyllin Monohydrate

The solubility was determined in different media by use of LC-UV chromatography. An excess amount of substance was weighed in vials and 0.5 ml of the medium was added. The substance was rotated in the specific medium at ambient temperature for 24 hours, followed by filtering the supernatant using a hydrophilic PVDF (Millipore Corp.) 0.22 μm filter. The samples were then diluted with a 1:1 mixture of mobile phase A and B (see below) and analyzed using an Xterra™ MS C₁₈, 50×2.1 mm column with UV detection at 288 nm. The mobile phase consisted of acetonitrile, water and trifluoroacetic acid, 5:95:0.1(A) and 99:1:0.1(B). The gradient profile was: 0-3 minutes with a linear increase of mobile phase B from 20% to 100% followed by 2 minutes with 100% B. The solubility was calculated from a calibration curve with accurately weighed amounts of the substance, dissolved and diluted to different concentrations with a 1:1 mixture of mobile phase A and B.

Solubility determinations were performed in 1% sodium dodecyl sulfate (SDS) of picropodophyllin monohydrate.

The solubility in 1% SDS after 24 hours rotation was 0.21 mg/ml for picropodophyllin monohydrate, which corresponds to 489 μM.

### Example 3

### Preparation of Picropodophyllin Polymorph A

3.02 g of picropodophyllin monohydrate was stored under vacuum in a desiccator next to a can of di-phosphorus pentaoxide over the weekend to give 2.90 g (theoretically 2.89 g) of picropodophyllin polymorph A.

### XRPD Peak Positions

### Picropodophyllin Polymorph A

### Refined 2θ Values:

6.9±0.2°2θ
7.9±0.2°2θ
9.2±0.2°2θ
9.7±0.2°2θ
15.0±0.2°2θ
16.7±0.2°2θ

### Example 4

### Biological Evaluation

A Phase I/II clinical trial with the IGF-1 receptor inhibitor picropodophyllin monohydrate was performed in patients with advanced, progressive cancer.

Ten patients with progressive non-small cell lung cancer (NSCLC) and with no treatment options were administered 390 or 520 mg picropodophyllin monohydrate twice-daily as monotherapy with a total duration of at least two weeks. The patients were assessed with imaging at the start of the study and thereafter every two months.

The median survival time of the ten patients with NSCLC was 42 weeks whereas the expected median survival time of such patients was less than 20 weeks. At cut-off, five of the patients were still alive and two of these patients had no detectable progression. Partial response was detected in one of these NSCLC patients according to RECIST criteria (Response Evaluation Criteria in Solid Tumors) following the treatment with picropodophyllin as hereinabove described.

1. A pharmaceutical composition comprising a pharmaceutically effective amount of picropodophyllin monohydrate, wherein the picropodophyllin monohydrate is characterized by an X-ray powder diffraction pattern exhibiting a peak at 6.9±0.2°2θ, in admixture with a pharmaceutically and pharmacologically acceptable carrier.

2. The pharmaceutical composition according to claim 1, wherein the picropodophyllin monohydrate is characterized by an X-ray powder diffraction pattern exhibiting peaks at 6.9 and 9.2±0.2°2θ.

3. The pharmaceutical composition according to claim 1, wherein the picropodophyllin monohydrate is characterized by an X-ray powder diffraction pattern exhibiting peaks at 6.9, 9.2, 13.7, 15.0, 20.6 and 21.5±0.2°2θ.

4. The pharmaceutical composition according to any one of claims 1 to 3, wherein the picropodophyllin monohydrate is substantially free from any other polymorph and/or other crystal and non-crystal forms of picropodophyllin.

5-14. (canceled)

15. A method for the treatment of cancer, whereby a therapeutically effective amount of picropodophyllin monohydrate characterized by an X-ray powder diffraction pattern exhibiting a peak at 6.9±0.2°2θ is administered to a patient in need of such treatment.

16. The method for the treatment of cancer according to claim 15, whereby a therapeutically effective amount of picropodophyllin monohydrate characterized by an X-ray powder diffraction pattern exhibiting peaks at 6.9 and 9.2±0.2 °2θ is administered to a patient in need of such treatment.

17. The method for the treatment of cancer according to claim 15, whereby a therapeutically effective amount of picropodophyllin monohydrate characterized by an X-ray powder diffraction pattern exhibiting peaks at characterized by an X-ray powder diffraction pattern exhibiting peaks at 6.9, 9.2, 13.7, 15.0, 20.6 and 21.5±0.2°2θ is administered to a patient in need of such treatment.

18. The method according to any one of claims 15-17, wherein said cancer is any one of lung cancer; breast cancer; head and neck cancer; gastrointestinal cancer; genitourinary cancer; gynecologic cancer; hematologic cancer; musculoskeletal cancer; skin cancer; brain and neurologic cancer; endocrine cancer; or eye cancer.

19. The method according to claim 18, wherein said cancer is non-small cell lung cancer (NSCLC).

20. The method according to claim 18, wherein said cancer is any one selected from small cell lung cancer; oral cancer; sinusoidal cancer; pharyngeal cancer; oesophageal cancer; stomach cancer; colon cancer; rectal cancer; gastrointestinal stromal tumor; liver cancer; pancreatic cancer; prostate cancer; bladder cancer; kidney cancer; ovarian cancer; cervical cancer; endometric cancer; uterine sarcoma; myeloid leukemia; lymphocytic leukemia; lymphomas; multiple myeloma; Ewing's sarcoma; osteosarcoma; soft tissue sarcoma; malignant melanoma; basal cell cancer; squamous cell cancer; Kaposi's sarcoma; glioma; glioblastoma; astrocytoma; medulloblastoma; craniopharyngeoma; neuroblastoma; adrenocortical cancer; paraganglioma; pheochromocytoma; thyroid cancer; retinoblastoma; or uveal melanoma.

21. A method for the treatment of psoriasis; restenosis after coronary angioplasty; diabetes mellitus type 2; nephropathy; eye diseases; rheumatoid arthritis; inflammatory bowel disease; multiple sclerosis; Alzheimer's disease; or graft rejection; whereby a therapeutically effective amount of picropodophyllin monohydrate characterized by an X-ray powder diffraction pattern exhibiting a peak at 6.9±0.2°2θ is administered to a patient in need of such treatment.

22. The method for the treatment of psoriasis; restenosis after coronary angioplasty; diabetes mellitus type 2; nephropathy; eye diseases; rheumatoid arthritis; inflammatory bowel disease; multiple sclerosis; Alzheimer's disease; or graft rejection according to claim 21, whereby a therapeutically effective amount of picropodophyllin monohydrate characterized by an X-ray powder diffraction pattern exhibiting peaks at 6.9 and 9.2±0.2°2θ is administered to a patient in need of such treatment.

23. The method for the treatment of psoriasis; restenosis after coronary angioplasty; diabetes mellitus type 2; nephropathy; eye diseases; rheumatoid arthritis; inflammatory bowel disease; multiple sclerosis; Alzheimer's disease; or graft rejection according to claim 21, whereby a therapeutically effective amount of picropodophyllin monohydrate characterized by an X-ray powder diffraction pattern exhibiting peaks at 6.9, 9.2, 13.7, 15.0, 20.6 and 21.5±0.2°2θ is administered to a patient in need of such treatment.

24. A pharmaceutical composition comprising a pharmaceutically effective amount of picropodophyllin polymorph A, wherein the picropodophyllin polymorph A is characterized by an X-ray powder diffraction pattern exhibiting a peak 6.9±0.2°2θ for use in therapy, in admixture with a pharmaceutically and pharmacologically acceptable carrier.

25. The pharmaceutical composition according to claim 24, wherein the picropodophyllin polymorph A is characterized by an X-ray powder diffraction pattern exhibiting peaks at 6.9 and 7.9±0.2°2θ.

26. The pharmaceutical composition according to claim 24, wherein the picropodophyllin polymorph A is characterized by an X-ray powder diffraction pattern exhibiting peaks at 6.9, 7.9, 9.2, 9.7, 15.0 and 16.7±0.2°2θ.

27-35. (canceled)

36. A method for the treatment of cancer, whereby a therapeutically effective amount of picropodophyllin polymorph A characterized by an X-ray powder diffraction pattern exhibiting a peak at 6.9±0.2°2θ, is administered to a patient in need of such treatment.

37. The method for the treatment of cancer according to claim 36, whereby a therapeutically effective amount of picropodophyllin polymorph A characterized by an X-ray powder diffraction pattern exhibiting a peak at 6.9 and 7.9±0.2°2θ is administered to a patient in need of such treatment.

38. The method for the treatment of cancer according to claim 36, whereby a therapeutically effective amount of picropodophyllin polymorph A characterized by an X-ray powder diffraction pattern exhibiting a peak at 6.9, 7.9, 9.2, 9.7, 15.0 and 16.7±0.2°2θ, is administered to a patient in need of such treatment.

39. The method according to any one of claims 36-38, wherein said cancer is any one of lung cancer; breast cancer; head and neck cancer; gastrointestinal cancer; genitourinary cancer; gynecologic cancer; hematologic cancer; musculoskeletal cancer; skin cancer; brain and neurologic cancer; endocrine cancer; or eye cancer.

40. The method according to claim 39, wherein said cancer is non-small cell lung cancer (NSCLC).

41. The method according to claim 40, wherein the non-small cell lung cancer (NSCLC) is adenocarcinoma, squamous or large-cell lung carcinoma.

42. A method for the treatment of psoriasis; restenosis after coronary angioplasty; diabetes mellitus type 2; nephropathy; eye diseases; rheumatoid arthritis; inflammatory bowel disease; multiple sclerosis; Alzheimer's disease; or graft rejection, whereby a therapeutically effective amount of picropodophyllin polymorph A characterized by an X-ray powder diffraction pattern exhibiting a peak at 6.9±0.2°2θ is administered to a patient in need of such treatment.

43. The method for the treatment of psoriasis; restenosis after coronary angioplasty; diabetes mellitus type 2; nephropathy; eye diseases; rheumatoid arthritis; inflammatory bowel disease; multiple sclerosis; Alzheimer's disease; or graft rejection according to claim 42, whereby a therapeutically effective amount of picropodophyllin polymorph A characterized by an X-ray powder diffraction pattern exhibiting a peak at 6.9 and 7.9±0.2° 2θ is administered to a patient in need of such treatment.

44. The method for the treatment of psoriasis; restenosis after coronary angioplasty; diabetes mellitus type 2; nephropathy; eye diseases; rheumatoid arthritis; inflammatory bowel disease; multiple sclerosis; Alzheimer's disease; or graft rejection; according to claim 42, whereby a therapeutically effective amount of picropodophyllin polymorph A characterized by an X-ray powder diffraction pattern exhibiting a peak at 6.9, 7.9, 9.2, 9.7, 15.0 and 16.7±0.2° 2θ is administered to a patient in need of such treatment.

45. The method according to claim 19, wherein the non-small cell lung cancer (NSCLC) is adenocarcinoma, squamous, or large-cell lung carcinoma.

46. The pharmaceutical composition according to any one of claims 24-26, wherein the picropodophyllin polymorph A is substantially free from any other polymorph and/or crystal and non-crystal forms of picropodophyllin.

47. The method according to claim 39, wherein said cancer is any one selected from small cell lung cancer; oral cancer; sinusoidal cancer; pharyngeal cancer; oesophageal cancer; stomach cancer; colon cancer; rectal cancer; gastrointestinal stromal tumor; liver cancer; pancreatic cancer; prostate cancer; bladder cancer; kidney cancer; ovarian cancer; cervical cancer; endometric cancer; uterine sarcoma; myeloid leukemia; lymphocytic leukemia; lymphomas; multiple myeloma; Ewing's sarcoma; osteosarcoma; soft tissue sarcoma; malignant melanoma; basal cell cancer; squamous cell cancer; Kaposi's sarcoma; glioma; glioblastoma; astrocytoma; medulloblastoma; craniopharyngeoma; neuroblastoma; adrenocortical cancer; paraganglioma; pheochromocytoma; thyroid cancer; retinoblastoma; or uveal melanoma.
